(19) United States
(12) Patent Application Publication
(10) Pub. No.: US 2026/0207157 A1
(43) Pub. Date: Jul. 23, 2026

(54) PHANTOM AND METHOD FOR REGISTRATION OF A FORM-DETECTING INSTRUMENT

(71) Applicant: Siemens Healthineers AG, Forchheim (DE)

(72) Inventors: Philipp Roser, Allston, MA (US); Marcus Pfister, Forchheim (DE)

(21) Appl. No.: 19/456,607

(22) Filed: Jan. 22, 2026

(30) Foreign Application Priority Data
Jan. 22, 2025 (DE) 10 2025 102 287.9

(51) Int. Cl. A61B 6/58; A61B 6/00

(52) U.S. Cl. CPC A61B 6/584; A61B 6/547

(57) ABSTRACT

A phantom and a method for mutual registration of an x-ray system and a form-detecting instrument are provided. In accordance with the method, the phantom is positioned in a recording area of the x-ray system. X-ray recordings of an x-ray feature of the phantom are then made from different recording angles. The form-detecting instrument is introduced into a form feature of the phantom. The form of the introduced form-detecting instrument is detected by the form-detecting instrument, where the position of the phantom during the detection of the form of the form-detecting instrument corresponds to its position during the x-ray recording. The form-detecting instrument and the x-ray system are mutually registered with the aid of the x-ray recordings and the detected form of the form-detecting instrument.

This application claims the benefit of German Patent Application No. DE 10 2025 102 287.9, filed on Jan. 22, 2025, which is hereby incorporated by reference in its entirety.

### BACKGROUND

The present embodiments relate to a phantom and to a method for mutual registration of coordinate systems of an x-ray system and a form-detecting instrument.

In minimally-invasive interventions using x-ray imaging, with C-Arm angiography systems, for example, therapies and diagnoses are carried out with instruments that are introduced into the body through small incisions (e.g., in the groin). The x-ray imaging is used in this case for navigation to a destination located within the body or within a blood vessel or hollow organ. With the help of x-ray imaging catheters and other instruments, as well as anatomical features, may be visualized. X-ray imaging, however, brings with it the disadvantage that the patient is exposed, as are the medical personnel, to a radiation dose.

In order to overcome the disadvantage of the radiation dose, the use of form-detecting instruments is known, which make possible a detection of their own shape or position without the use of x-ray radiation. The form detection may be undertaken, for example, by fiber-optic 3D form sampling based on light guidance. To determine the form, for example, the modulation of the light guidance in the optical fiber is used. The use of this technique is known, for example, for visualizing guide wires. Form detection is undertaken in this case without x-ray imaging and thus without any radiation dose. From the detected spatial form, when the position of the starting point of the optical fiber is known, the spatial position in the fiber may be derived. Further approaches free from x-ray radiation are known (e.g., based on electromagnetic tracking), in which electromagnetic fields are used for determining the spatial position. The spatial form of the tracked instrument may be derived from the position or positions determined. What is basically meant below by the term form-detecting is that a spatial form or position of an instrument may be detected without the use of x-ray radiation.

Systems for determination of form and position normally use their own spatial coordinate system. In fiber-optic 3D-form sampling, for example, anchoring of the coordinate system of the fiber-optic system in the vicinity of the relevant destination location of the position determination and in a suitable alignment to the destination location is known, for example. X-ray systems normally likewise use their own spatial coordinate system. The coordinate system of an x-ray system is anchored as a rule in relation to x-ray emitter or x-ray detector. The form and position information from form-detecting systems free from x-ray radiation (e.g., fiber-optic systems) may be able to be used sensibly as part of procedures using x-ray imaging. To do this, the definition of a common coordinate system of the form-detecting system and of the x-ray system or the definition of a spatial transformation specification between the two coordinate systems is to be provided. Such a transformation specification is also referred to as the registration of the two coordinate systems or the registration of the x-ray system and the form-detecting instrument. The terms transformation and registration below may be a spatial transformation or spatial registration. The term registration below may be that a transformation specification is established between coordinate systems or that a coordinate system is transformed into another coordinate system, or that coordinate systems are transformed into a common single coordinate system.

An imaging system (e.g., a C-arm x-ray system) may, for example, be calibrated and registered using a spiral phantom. A spiral phantom for x-ray-registration may have a series of markers visible to x-rays, or also x-ray marks. The x-ray markers may be arranged in or on the phantom so that the arrangement extends on all three spatial directions (e.g., is not linear or planar). The x-ray markers may be configured as spheres visible to x-rays. The x-ray markers are arranged asymmetrically and may be different from one another, so that, with the aid of one or just a few x-ray recordings, the x-ray marker position and orientation of the x-ray markers and thus of the phantom in relation to the x-ray system may be determined on a unique basis.

The registration of the coordinate systems of an x-ray system and a form-detecting instrument is basically known in the prior art. For registration of the two coordinate systems, as a rule, two x-ray images of the form-detecting instrument are recorded. With the aid of the two x-ray images, the position of the instrument in relation to the x-ray system is reconstructed. The reconstructed position is then registered with the position information of the form-detecting instrument. This method requires the recording of two or more x-ray images and is thus complex and time-consuming. The accuracy of this method depends on the accuracy with which the position of the instrument can be reconstructed from the x-ray images.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, the known methods may be improved.

The present embodiments include a phantom and a method that are based on registration features that are able to be detected both for an x-ray system and also for a form-detecting instrument. The present embodiments thus give a fast, precise, low-effort and radiation-free or reduced-radiation method for registration of the coordinate system of an x-ray system and of a form-detecting instrument.

The phantom of the present embodiments for mutual registration of a form-detecting instrument and an x-ray system has an x-ray feature that is configured to make possible a registration of the phantom in the coordinate system of the x-ray system with the aid of x-ray recordings of the phantom by the x-ray system. The phantom has a form feature that is configured to make possible a registration of the phantom in the coordinate system of the form-detecting instrument with a form detection via the form-detecting instrument. The form feature is configured to introduce the form-detecting instrument into the form feature and thereby to bring the form feature into a registration form that extends in all three spatial directions.

The x-ray feature may, for example, involve a known arrangement of calibration phantoms of x-ray markers. The form feature is configured such that it makes it possible to position a form-detecting instrument in the spatial area of the x-ray feature or in the spatial environment about the x-ray feature in a form and position predetermined by the form feature. The form feature is configured to introduce the form-detecting instrument into the form feature and thereby bring the form feature into a registration form that extends in all three spatial directions.

The phantom in accordance with the present embodiments may be regarded as a combined phantom that integrates the individual phantoms both for the x-ray system and also for the form-detecting instrument. The relationship of the individual phantoms to each other is predetermined as fixed by the phantom and, in this way, makes possible the registration of the coordinate system of the x-ray system and of the form-detecting instrument with one another.

The phantom involves an apparatus that brings the form-detecting instrument into a registration position and registration form. The form-detecting instrument may, for example, be a guide wire, a catheter, an endoscope, a laparoscope, or another flexibly deformable instrument. The registration position may, for example, be reached when the form-detecting instrument is completely introduced right up to its end into the form feature. The registration position is a position of the instrument, in which the instrument may be correctly registered. The relative spatial position of the form feature in relation to the x-ray feature of the phantom is predetermined in a constructively fixed manner by the phantom. The relative spatial position of the x-ray feature in relation to the x-ray system may be established with the aid of one or more x-ray recordings. The relative spatial position of the form-detecting instrument introduced into the form feature in relation to the x-ray system may be established, for example, with the aid of the one or more x-ray recordings. The relative spatial position of the x-ray feature in relation to the x-ray system may, for example, also be derived from the known relative spatial position of the form feature in relation to the x-ray feature as soon as the x-ray feature is registered.

The registration method may be carried out on the x-ray system side or on the form-detecting instrument side, because both the x-ray system and also the form-detecting instrument each have a coordinate system available. The x-ray system includes a controller facility (e.g., a controller), and the form-detecting instrument includes a control facility (e.g., a controller). The controller facilities each work with their own coordinate system. One of the two facilities or both of the two facilities may carry out the registration method.

The present embodiments enable the effort and the time required for the registration of the coordinate system to be reduced since the x-ray system and the form-detecting instrument may be mutually registered by the phantom with the aid of the same one or more x-ray recordings. Further, the phantom in the shape of the form features predetermines a suitable three-dimensional registration form for the registration of the form-detecting instrument. The present embodiments enable the precision of the registration to be improved, since both systems may be registered with the aid of the same x-ray recording or x-ray recordings, so that unwanted relative movements of x-ray system and form-detecting instrument between registration x-ray recordings are avoided. The present embodiments enable the effort to be reduced, since no additional registration x-ray recordings for separate registration processes for the x-ray system and the form-detecting instrument are to be provided.

A development of the present embodiments makes provision for the phantom to have an x-ray feature in the shape of markers visible to x-rays, that are arranged on or in the phantom, and the arrangement of which extends in all three spatial directions. The registration of phantoms with markers visible to x-rays is known. Powerful registration methods that the reader may refer back to for this purpose are available.

A development of the present embodiments makes provision for the form feature to be configured as a tube. The tube may, for example, have a round cross section. Such a tube is well suited for introduction, for example, of an endoscope, laparoscope, catheter, or guide wire. For example, the form of a tube with a round cross section is similar to the basic form of a blood vessel and is therefore especially good for introduction of an intravascular instrument, such as, for example, a guide wire or catheter. Further, a tube (e.g., a tube with a round cross section) is easy to produce and simple to integrate into a phantom.

A development of the present embodiments makes provision for the phantom additionally to have an optical feature, with the aid of which a registration of the phantom in the coordinate system of an optical camera arrangement is possible. With this, an additional optical registration is made possible, through which the accuracy of the registration may be increased. Further, the optical registration may also be carried out instead of a registration based on x-ray recordings, through which the radiation dose and the outlay for x-ray recordings in the registration method as a whole may be reduced. However, the optical feature is to be registered for this purpose in the coordinate system of the x-ray system. Thus, a saving in radiation dose and effort is possible when the optical feature is not registered with the x-ray system with the aid of x-ray recordings, but, for example, likewise based on an optical recording. For this, the x-ray system may, for example, be equipped with an additional optical camera arrangement that is permanently connected to the x-ray system and is registered with the system in advance. Thus, when the x-ray system has an optical camera arrangement registered with the system in advance, the optical feature of the phantom may be registered with this without x-ray recordings having to be made for this.

A development of the present embodiments makes provision for the phantom to have a detection facility with which it is possible to recognize when the form-detecting instrument is being introduced into the phantom. When the detection facility recognizes that the instrument is introduced into the phantom, the detection facility generates a registration signal.

The detection facility is configured so that it is possible to recognize when the instrument is introduced at least up to a specific position. This position is chosen so that it is suitable for a mutual registration of form-detecting instrument and x-ray system. When the detection facility recognizes that the instrument has reached this position, the detection facility generates a registration signal. The detection facility may, for example, be configured as a contact sensor or touch sensor, or as a capacitive or optical sensor. In such cases, conventional and commercially-available sensors may be involved, which are inexpensive, reliable, and low-cost. In response to the generation of the registration signal, the mutual registration of instrument and x-ray system may be carried out by a computer-implemented registration method.

The registration method may be carried out on the x-ray system side or the form-detecting instrument side, because both the x-ray system and also the form-detecting instrument each have a coordinate system available. The x-ray system includes a controller facility, and the form-detecting instrument includes a control facility. One of the two facilities or both of the facilities in parallel may carry out the registration method. The registration method may also be carried out by a further, other computing facility. Accordingly, the registration signal is transferred by the detection facility to the controller facility of the x-ray system, to the control facility of the form-detecting instrument, or to the further computing facility. As soon as the registration signal is received, the registration method is started in the receiving facility or facilities.

The autonomous starting of the registration method of the present embodiments enables the effort and time required for the registration of the coordinate systems to be reduced. The detection of the position of the form-detecting instrument in the phantom suitable for a mutual registration enables the precision of the registration to be improved since, with the suitable registration position known in advance, the precise relative position of the instrument in relation to the x-ray system is known exactly.

In accordance with the method of the present embodiments, a phantom in accordance with the present embodiments is positioned in the recording area of the x-ray system. Then, x-ray recordings of the x-ray feature of the phantom are recorded by the x-ray system from different recording angles. During the x-ray recordings, the position of the phantom remains unchanged. Further, the form-detecting instrument is introduced into the form feature of the phantom. The form-detecting instrument may be introduced into the phantom before the recording of the x-ray recordings, so that the form-detecting instrument is mapped in the x-ray recordings and may thus provide additional information for the registration method. It is, however, also possible to introduce the form-detecting instrument into the phantom only after the recording of the x-ray recordings or independently of the recording of the x-ray recordings, if no x-ray recordings of the form-detecting instrument are needed for the further registration method. This is advantageous, for example, when the form-detecting instrument is sensitive to x-ray radiation. The form of the introduced form-detecting instrument is detected by the form-detecting instrument, where the position of the phantom during the detection of the form of the form-detecting instrument corresponds to the position of the phantom during the recording of the x-ray recordings. Subsequently, the form-detecting instrument and the x-ray system are mutually registered with the aid of the x-ray recordings and the detected form of the form-detecting instrument.

In accordance with the present embodiments, the mutual registration of the form-detecting instrument and the x-ray system or the mutual registration of their coordinate systems is undertaken by using a single phantom. This saves time and money, since only a single phantom is to be used and this only has to be placed one single time. Further, the situation in which only one phantom is used in a position that stays the same makes possible the detection of the form of the form-detecting instrument by the form-detecting instrument and the position of the phantom by the x-ray system at the same time. In addition, the form of the form-detecting instrument may also be detected by the x-ray system at the same time if the form-detecting instrument is already introduced into the phantom at the point of the x-ray recording or x-ray recordings. In this way, a renewed positioning of the phantom or a separate carrying-out of the two form detection steps may be avoided.

In accordance with a development of the method, at least two x-ray recordings of the form-detecting instrument are recorded using recording angles that differ from one another by at least 30 degrees (e.g., orthogonal to one another). The position of the phantom and the form-detecting instrument remains unchanged during the x-ray recordings. Then, the form of the form-detecting instrument is reconstructed from the at least two x-ray recordings. A reconstruction based on a small number of x-ray recordings allows at least the establishing of position and course of the form-detecting instrument, even if there is no complete three-dimensional reconstruction. Then, the form-detecting instrument and the x-ray system or their coordinate systems are mutually registered with the aid of the reconstructed and the detected form of the form-detecting instrument.

By x-ray recordings being used both for detection of the x-ray feature of the phantom and also for detection of the form of the form-detecting instrument, an especially reliable registration is able to be achieved in relation to the x-ray system. Further, the detection of the x-ray features and the form of the form-detecting instrument may be undertaken with the aid of the same x-ray recordings, so that time and effort for the recording of additional x-ray recordings may be saved. In combination with the form detected by the form-detecting instrument itself, two different items of information relating to the form of the form-detecting instrument are thus available for the registration method, whereby the accuracy and the robustness of the method may be enhanced.

In accordance with a development of the method, a phantom model that includes a spatial form model of the form feature is received. The model may be formed based on widely-used model systems (e.g., may involve a 3D mesh model). The form model of the form feature included by the phantom model is provided by being read out from the phantom model or taken or extracted from the phantom model. Then, the form-detecting instrument and the x-ray system or their coordinate systems are mutually registered with the aid of the form model and the detected form of the form-detecting instrument.

The form of the form feature including position and course of the same is known with a high level of accuracy from the construction of the phantom known in advance. As a consequence, the form of the form-detecting instrument introduced into the form feature, which is characterized by the form of the form feature, is known with a high level of accuracy and is not subject to any inaccuracies caused by a reconstruction of the form, which for example may be undertaken with the aid of x-ray recordings. This enables the accuracy of the registration method to be increased. Further, additional method acts, outlay, and radiation dose for reconstruction of the form of the form-detecting instrument with the aid of x-ray recordings are avoided. This enables the effort involved in the registration method to be reduced and the registration method to be carried out with lower time outlay and with higher accuracy.

Further embodiments and advantages emerge from the dependent claims as well as from the description given below of example embodiments with the aid of figures.

### BRIEF DESCRIPTION OF THE DRAWINGS

FIG. 1 shows an x-ray system and form-detecting instrument with a phantom;

FIG. 2 shows a phantom in accordance with an embodiment;

FIG. 3 shows a phantom in accordance with an embodiment in another view;

FIG. 4 shows a phantom in accordance with an embodiment in an overhead view; and

FIG. 5 shows a phantom in accordance with an embodiment with optical markers.

### DETAILED DESCRIPTION

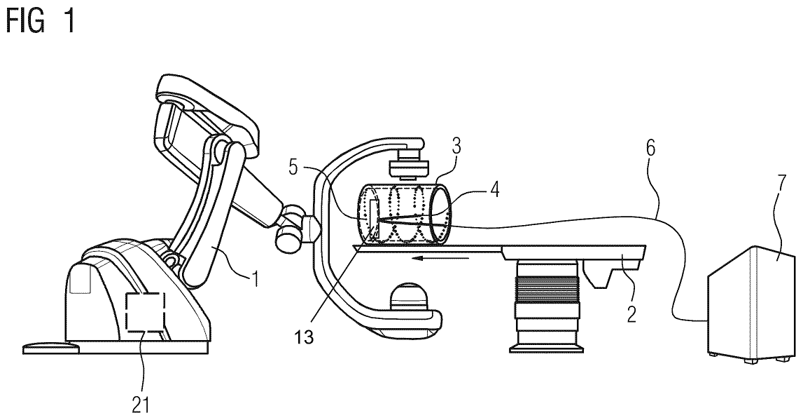

Shown schematically in FIG. 1 is an x-ray system 1. The x-ray system 1 is controlled by a controller facility 21 (e.g., a controller). Assigned to the x-ray system 1 is a patient couch 2. Objects or patients that are positioned on the patient couch 2 may be recorded with the x-ray system 1.

Also shown schematically in the diagram is a form-detecting instrument 6. The form-detecting instrument 6 is controlled by a control facility 7 in order to generate sensor data and evaluate the sensor data. The form-detecting instrument 6 may, for example, involve an instrument that has an optical form-detecting fiber. Such fibers are known and allow the form and the course of the fibers and thereby of the form-detecting instrument 6 to be detected. The evaluation of the sensor data that is generated by the form-detecting fibers enables the control facility 7 to establish the current form and the current course of the fibers and thus of the form-detecting instrument 6.

Positioned in an imaging area of the x-ray system 1 on the patient couch 2 is a phantom 3 of the present embodiments. The phantom 3 includes as a form feature a tube 4, into which the form-detecting instrument 6 is completely introduced. Completely introduced in this case may be that the form-detecting instrument 6 is introduced up to an end of the tube 4. The end of the tube 4 stops the form-detecting instrument 6 being pushed any further forward. The phantom 3 also includes a series of x-ray features that are configured as x-ray markers 5. The x-ray markers 5, like the tube 4, are each arranged within the phantom 3 extended in all three spatial directions. The x-ray markers 5 may also be of different sizes and may differ in their transparency for x-rays, in order to make it possible for the x-ray markers 5 to be distinguished in x-ray recordings. Arrangement and visibility of the x-ray markers 5 facilitate a detection of positions of the x-ray markers 5 in x-ray recordings and, through this, facilitates or improves the establishment of the respective three-dimensional position in space.

The phantom 3 is conventionally configured with respect to the x-ray markers 5. Such phantoms are known and allow a precise registration between x-ray system and phantom. In addition, the phantom 3 has the tube 4, with the aid of which the form-detecting instrument 6 is brought into a three-dimensionally extended form and position. Since the tube 4 is located in the imaging area of the x-ray system 1, a mutual registration of the x-ray system 1 and the form-detecting instrument 6 is made possible.

For the mutual registration, one or more x-ray recordings of the phantom 3 along with the form-detecting instrument 6 are recorded. If a number of x-ray recordings are recorded, these may differ with respect to their recording angle. If the recording angle differs by at least 30 degrees, spatial positions of the mapped objects may be reconstructed from the x-ray recordings. An especially accurate reconstruction is possible with a difference of 90 degrees. From the x-ray recording or recordings, the x-ray system 1 is registered with the phantom 3 with the aid of the x-ray markers 5. Thus, a registration is available for the x-ray system 1 and the imaging area.

At the same time, the form-detecting instrument 6 may be reconstructed or at least symbolically reconstructed from a number of the x-ray recordings, of which the recording angle differs by at least 30 degrees. Symbolic reconstruction may, in this case, be a reconstruction of position and course of the form-detecting instrument 6, which is not, however, a complete three-dimensional representation of the form-detecting instrument 6. With the reconstruction, position and course of the form-detecting instrument 6 are available in the coordinate system of the x-ray system 1.

As an alternative or in addition to this, position and course of the form-detecting instrument 6 in the coordinate system of the x-ray system 1 are also established from the construction of the phantom 3 known beforehand. The form-detecting instrument 6 assumes the constructively determined and physically fixed predetermined position and course of the tube 4 in the tube 4. Position and course of the tube 4 in the phantom 3 may be assumed as known beforehand. This also enables position and course of the tube 4 in relation to the x-ray markers 5 to be assumed as known beforehand. Thus, position and course of the form-detecting instrument 6 introduced into the tube 4 in relation to the x-ray markers 5 and thus indirectly in relation to the x-ray system 1 are also known.

For mutual registration, position and course of the form-detecting instrument 6, in addition to the coordinate system of the x-ray system 1, are also established in the coordinate system of the form-detecting instrument 6. For this purpose, position and course of the form-detecting instrument 6 are established by the control facility 7. The establishment of position and course of the form-detecting instrument 6 may be undertaken, for example, at the same time as the x-ray recording or recordings that are recorded of the phantom 3 for registration purposes. The establishment may also be undertaken at a different time. Then, it is merely to be provided that the position of the phantom 3, including its orientation, remains unchanged. In order to provide that the phantom 3 does not move, the phantom 3 may be permanently connected, for example, with a holder to the patient couch 2.

Then, with the aid of position and course of the form-detecting instrument 6 in the coordinate system of the x-ray system 1 and in the coordinate system of the form-detecting instrument 6, the mutual registration may be undertaken. For this, for example, information about the detected form of the control facility 7 may be transferred to the controller facility 21. Conversely, information about the detected form may be transferred from the controller facility 21 and the control facility 7, or information about the respective form may be transferred by the control facility 7 and by the controller facility 21 to a separate computer. The registration may then be carried out using known registration methods by the control facility 7, the controller facility 21, or the separate computer.

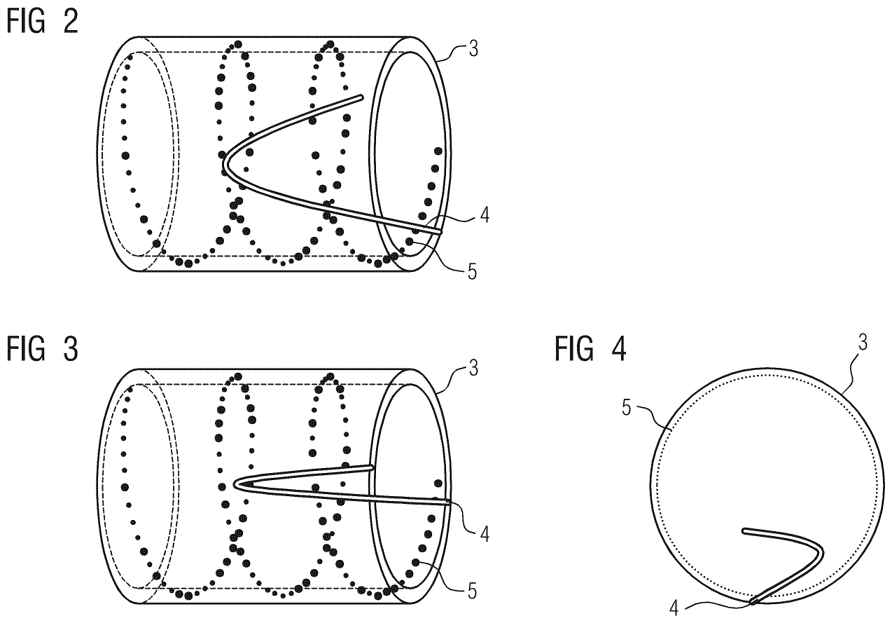

Shown schematically in FIG. 2 is the phantom 3 of the present embodiments. The x-ray markers 5 are of different sizes, by which the x-ray markers 5 are distinguished from one another. The x-ray markers 5 are arranged in a spiral shape running around close to an outer side of the phantom 3. Arrangement and size of the x-ray markers 5 allow the position and orientation of the phantom 3 to be established with the aid of two or more x-ray recordings. To do this, the x-ray recordings are recorded at recording angles that differ from one another by at least 30 degrees.

The phantom 3 also has a tube 4, into which a form-detecting instrument 6 may be introduced. The tube 4 is matched to the form-detecting instrument 6 (e.g., through a suitable cross section and diameter), so that the tube 4 may accept the form-detecting instrument 6 into itself. Since the tube 4 is configured along its length in the form of a tube, the tube 4 may accept an elongated, hose-like form-detecting instrument 6 into itself. The form of the form-detecting instrument 6 in this case, with respect to position and course, assumes a form similar to that of the tube 4. Similar may, in this case, be that the form of the form-detecting instrument 6, when introduced into the tube 4, approximately matches the form of the tube 4.

The exactness of the match essentially depends in this case on the play between the form-detecting instrument 6 and the tube 4. The play may make possible an easy and resistance-free introduction of the instrument 6. For example, if the form of the tube 4 known in advance is to be employed for the registration of the form-detecting instrument 6, the play may be as small as possible to provide a high precision. In this case, when the play is set, the accuracy of the form detection by the instrument may be taken into account; if the instrument makes possible a form detection with millimeter accuracy, the play may also be set in the millimeter range. If the form of the tube 4 is not to be employed for the registration of the form-detecting instrument 6, the diameter of the tube 4 may be chosen as comparatively large in order to allow the tube 4 to accept the largest range of instruments 6 into the tube 4.

Shown schematically in FIG. 3 is the phantom 3 of the present embodiments in another perspective. In the altered perspective, the tube 4 especially is presented differently. The diagram serves to illustrate the three-dimensional extent (e.g., of the tube 4).

Shown schematically in FIG. 4 is the phantom 3 of the present embodiments in yet another perspective (e.g., in an overhead view). The diagram serves to illustrate the arrangement of the x-ray markers 5 in the outer area of the phantom 3 and also the spatial extent of the tube 4.

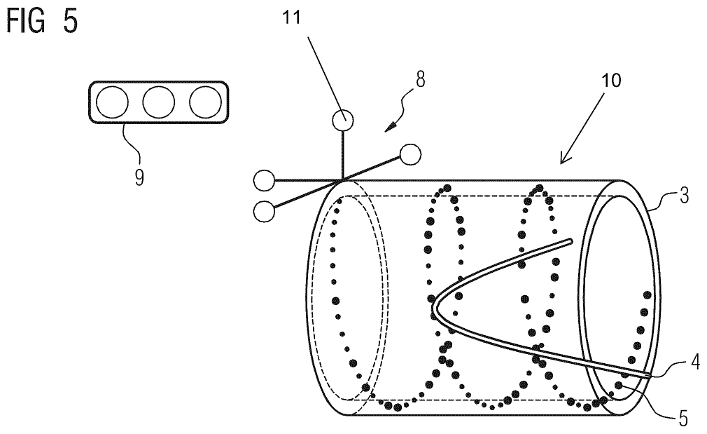

Shown schematically in FIG. 5 is a phantom 10 of the present embodiments that has an additional optical feature in the shape of an optical marker facility 8. As regards the x-ray markers 5 and the tube 4, the description above is referred to. The additional marker facility 8 includes a series of optical markers 11 and is attached at a fixed location on the phantom 10. The optical markers 11 of the additional marker facility 8 may be detected by an optical camera system 9. The camera system 9 may be based on a normal optical camera, for example, or on a depth camera. The optical markers 11 of the marker facility 8 are arranged spatially so that the spatial position and orientation of the marker facility 8 may be established with the images detected with the aid of the camera. To this extent, the optical marker facility 8 along with the camera system 9 represents a known optical tracking system, and the detection of position and orientation is undertaken in accordance with known methods.

The optical marker facility 8 may be attached to the phantom 10 both permanently and also detachably. What is to be provided is merely that the optical marker facility 8 remains in the same position on the phantom 10 during the x-ray recordings 8 and the camera detection. Instead of or in addition to the optical marker facility 8, optical markers may also be arranged directly on the phantom 10. Known embodiments may be used (e.g., circles, reticles, or chess board patterns).

The optical marker facility 8 or the optical tracking of the phantom 10 allows its position and orientation to be established in the coordinate system of the optical tracking system. In order to establish position and orientation of the phantom 10 in the coordinate system of the x-ray system, the optical tracking system may also establish its position and orientation using corresponding optical markers on the x-ray system 1. Thereby, with the aid of the position and orientation of the phantom 10 and of the x-ray system 1 established by the tracking system, a mutual registration of phantom 10 and x-ray system 1 may be carried out. Using the previously known position and the previously known course of the tube 4 in the phantom 10, position and course of a form-detecting instrument introduced into the tube 4 may likewise be established in the coordinate system of the optical tracking system and registered with the coordinate system of the x-ray system 1.

Referring to FIG. 1, the phantom 3 may include, in accordance with an embodiment, a detection facility 13 (e.g., a detector). With regard to the x-ray markers 5 and the tube 4, the reader is referred to the description given above. Using the detection facility 13, it is possible to detect whether an instrument has been completely introduced into the tube 4. As soon as the instrument is completely introduced into the tube 4, the instrument has assumed a position and form that is suitable for the registration of the coordinate system of the instrument with that of the x-ray system 1. When the detection facility 13 detects that an instrument is completely introduced into the tube 4 (e.g., has assumed a position and form suitable for a registration), the detection facility 13 generates a registration signal. The detection facility 13 may, for example, include a contact sensor or a touch sensor arranged in the tube 4, which reacts when an instrument touches the end of the tube 4. The detection facility 13 may, for example, also include an optical sensor that makes it possible to detect optically when an instrument is pushed forward into the end area of the tube 4.

The registration signal is transferred to that computing facility or those computing facilities that is or are carrying out the registration method. The registration signal is thus transferred to the controller facility 21, the control facility 7, or to a separate computing facility. As soon as the computing facility or the computing facilities concerned receive the registration signal, the carrying-out of the registration method is initiated. To carry out the registration method, position and course of the instrument in the coordinate system of the x-ray system 1 and also in the coordinate system of the instrument are received by the computing units concerned. With aid of this information, the mutual registration may be carried out in the known manner. The detection facility 13 thereby makes it possible to start the registration method automatically, as soon as the instrument is introduced into the tube 4 of the phantom 3.

In the above description, independent of the grammatical term usage, individuals with male, female, or other gender identities are included within the term.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A phantom for mutual registration of a form-detecting instrument and an x-ray system, the phantom comprising:
an x-ray feature that is configured to make possible a registration of the phantom in a coordinate system of the x-ray system with the aid of x-ray recordings of the phantom by the x-ray system; and
a form feature that is configured to make possible a registration of the phantom in a coordinate system of the form-detecting instrument with the aid of a form detection by the form-detecting instrument,
wherein the form feature is configured to introduce the form-detecting instrument into the form feature and through this, bring the form-detecting instrument into a registration form that extends in all three spatial directions.

2. The phantom of claim 1, wherein the x-ray feature is configured as a marker visible to x-rays, that is arranged on or in the phantom, and of which the arrangement extends in all three spatial directions.

3. The phantom of claim 1, wherein the form feature is configured as a tube.

4. The phantom of claim 1, further comprising an optical feature that is configured to make possible a registration of the phantom in a coordinate system of an optical camera arrangement.

5. The phantom of claim 1, further comprising a detector that is configured to recognize when the form-detecting instrument is being introduced into the phantom,
wherein a registration signal is generatable using the detector when the detector recognizes that the form-detecting instrument is introduced into the phantom.

6. A method for mutual registration of a form-detecting instrument and an x-ray system, the method comprising:
positioning a phantom in a recording area of the x-ray system;
recording x-ray recordings of an x-ray feature of the phantom by the x-ray system from different recording angles, wherein a position of the phantom is unchanged during the x-ray recordings;
introducing the form-detecting instrument into a form feature of the phantom;
detecting a form of the form-detecting instrument by the form-detecting instrument, wherein the position of the phantom corresponds to the position of the phantom during the x-ray recordings; and
mutual registering the form-detecting instrument and the x-ray system with the aid of the x-ray recordings and the detected form of the form-detecting instrument.

7. The method of claim 6, wherein the x-ray feature is configured to make possible a registration of the phantom in a coordinate system of the x-ray system with the aid of the x-ray recordings of the phantom by the x-ray system;
wherein the form feature is configured to make possible a registration of the phantom in a coordinate system of the form-detecting instrument with the aid of the form detecting by the form-detecting instrument, and
wherein the form feature is configured to introduce the form-detecting instrument into the form feature and through this, bring the form-detecting instrument into a registration form that extends in all three spatial directions.

8. The method of claim 6, further comprising:
recording at least two x-ray recordings of the form-detecting instrument, of which recording angles differ by at least thirty degrees from one another, wherein the position of the phantom and of the form-detecting instrument is unchanged during the x-ray recordings;
reconstructing the form of the form-detecting instrument from the at least two x-ray recordings; and
mutual registering the form-detecting instrument and the x-ray system with the aid of the reconstructed form of the form-detecting instrument and the detected form of the form-detecting instrument.

9. The method of claim 8, wherein the recording angles are orthogonal to each other.

10. The method of claim 6, further comprising:
receiving a phantom model that comprises a spatial form model of the form feature,
providing the form model; and
mutual registering the form-detecting instrument and the x-ray system with the aid of the form of the form model and the detected form of the form-detecting instrument.
